(19) United States
(12) Patent Application Publication
(10) Pub. No.: US 2024/0423844 A1
(43) Pub. Date: Dec. 26, 2024

(54) TOPICAL WOUND DRESSINGS AND METHODS FOR USE THEREOF

(71) Applicants: Shahryar AHMADI, Huntington Beach, CA (US); Shahin S. Rad, Encino, CA (US)

(72) Inventors: Shahryar AHMADI, Huntington Beach, CA (US); Shahin S. Rad, Encino, CA (US)

(21) Appl. No.: 18/750,626

(22) Filed: Jun. 21, 2024

Related U.S. Application Data
(60) Provisional application No. 63/522,231, filed on Jun. 21, 2023.

(51) Int. Cl. A61F 13/0246; A61F 13/01; A61F 13/0206

(52) U.S. Cl. CPC A61F 13/0253; A61F 13/01008; A61F 13/0206

(57) ABSTRACT

Wound dressings for local application of therapeutic agents to a wound site include multilayer dressings and topical compositions for direct application to the skin, both comprising at least one therapeutic agent that is cannabidiol. The inventive wound dressings deliver and retain therapeutic agents locally at the wound site, while limiting or eliminating systemic absorption. Multilayer dressings of the invention also provide a means to add or replenish the therapeutic agent without changing the need to change the dressings.

### FIELD OF THE INVENTION

The invention is directed to wound dressings for promoting wound healing that contain at least one therapeutic component, cannabidiol (CBD). The inventive wound dressings are formulated to improve wound healing by enhancing local effects of therapeutic agents at the dermal level, while eliminating or significantly reducing systemic absorption and the frequency of dressing changes. Inventive wound dressings include topical, adhesive dressings for application to the skin, configured with multiple sections or layers, at least one of which incorporates CBD alone or in combination with other therapeutic agents, and topical compositions for direct application to the skin comprising CBD as the therapeutic agent alone or in combination with other therapeutic agents.

### BACKGROUND OF THE INVENTION

### Wound Assessment and Classification

Wounds form when tissues such as skin, membranes, bones and organs are damaged or injured by some form of trauma. Wounds may be classified in different ways. Traditional wound classifications and assessments are well described in literature. Proper assessment and classification of wounds is important to the diagnosis, management, and selection of the correct treatment based on wound type. Wounds may be evaluated in terms of risk factors that affect healing, the presence or absence of infection, physical location, size, appearance, and stage of healing. The presence of edema, hematoma, perfusion and the status of tissues and the rate of healing in relation to the date of injury are also important factors to assess.

Depending on healing time, a wound may be classified as acute or chronic. Acute wounds are caused by sudden trauma and usually progress through normal stages of healing. Common acute wound types include incisions, lacerations, abrasions, punctures, or contusions. Acute wounds usually heal completely with no complications in the expected time frame and may be referred to as non-chronic wounds. On the other hand, chronic wounds are characterized by delays in healing time and/or complications and may be associated with underlying health conditions or impaired wound healing mechanisms. Common types of chronic wounds are pressure ulcers (sores), diabetic foot ulcers, venous ulcers, arterial ulcers, neuropathic ulcers, among many others.

Wounds may also be classified as open or closed. Open wounds expose tissue, bone or organs to the external environment, while closed wounds do not expose underlying tissue, bone or organs to the external environment.

Surgical wounds are incision type wounds resulting from surgical procedures. They are commonly further classified based on their level of contamination and risk of infection as clean wounds, clean-contaminated wounds, contaminated, or infected wounds.

Some wounds are highly unique and have underlying causes that require specific management approaches. These wounds include burns caused by thermal, chemical or electrical injury to the skin and underlying tissues, radiation ulcers, fungal infections, and ischemic ulcers.

### Wound Healing

Wound healing starts almost immediately after an injury. Wound healing is the body's repair response to any number of different injuries caused by trauma, surgery, burns, disease states, and the like. Wound healing is one of the most complex processes in the human body and involves the spatial and temporal synchronization of a variety of cell types having distinct roles in healing stages. As is generally accepted, wound healing is a natural cellular process that occurs in stages that include: homeostasis, inflammation, proliferation and maturation or remodeling. While these stages usually progress in a linear order, the duration of any stage may vary for different wound types, and healing progress depends on internal and external patient conditions. Although a variety of mechanisms have been studied, what is clear is that healing is related to the degree of the injury and cellular activity responds to produce the amount of healing required. E.g., Wilkinson H N, Hardman M J. 2020 Wound healing: cellular mechanisms and pathological outcomes. Open Biol. 10:200223.http://dx.doi.org/10.1098/rsob.200223.

### Wound Dressings

Wound healing is facilitated by proper cleaning and dressing of wounds, which is essential to prevent infection and additional injury and to promote healing. Wound dressings are commonly utilized to promote healing of the wound through controlling of exudate, protecting the wound from contamination and further injury, preventing bacterial contamination or overgrowth, and maintaining a moist wound environment. Wound dressings take many forms and include without limitation bandages, patches, films, sheets, gauzes, woven and non-woven fabrics, absorbent materials, hemostats, glues, sealants, gels, creams, ointments, salves, pastes, sprays, foams, sponges, occlusive dressings, or any other device or composition used to facilitate wound healing. Various forms of wound dressings may be combined for functional advantages.

Historically, many diverse materials have been used to treat wounds by absorbing and controlling fluids (“exudate”) released from a wound site. Exudate is a byproduct of vasodilation during the inflammatory stage. Management of exudate depends on the amount of exudate, location and exudate composition. Depending on the volume of exudate, more frequent dressing changes may be required. A wide variety of wound care products for receiving, absorbing, and retaining exudate, while maintaining a moist, clean environment for the wound, are commercially available. Suitable materials for use as absorbent dressings to absorb and control exudate and/or maintaining a moist wound environment include without limitation cotton, woven and non-woven fabrics, gauzes, foams, sponges, polymer films, particulate polymers, fibrous polymers, hydrogels, hydrocolloids, and mixtures thereof. The purpose of these materials is to remove excess exudate and toxic components, to serve as a barrier to protect the wound from injury or secondary bacterial infection and to hydrate the wound. However, not all absorbent materials provide or allow for a moist wound environment. Maintaining a moist wound environment is known to facilitate healing. As such, occlusive or moisture retentive dressings have gained increasing acceptance in treating wounds and are deemed to provide optimal conditions for wound healing.

For wounds without little to no exudate, absorbent materials may not be required in the dressing. In such cases, occlusive topical dressings are used to protect and soothe the wound, create a barrier to air exposure, bacterial contamination, or further damage, and maintain a moist environment through hydration. While no wound dressing can provide an absolute sealing of a wound, the non-absorbent nature of occlusive dressings may be sufficient in many situations. Suitable occlusive topical dressings are known in the art and include topical compositions such as creams, gels, ointments, pastes, salves and waxes, as well as polymer films, polymer foams, hydrocolloids, and hydrogel dressings. Occlusive dressings such as polymer foams may also be used to absorb exudate.

Ideally, a wound dressing should allow for fewer dressing changes to allow the wound to be left undisturbed. When wounds are left undisturbed in a moist environment, they can heal at a faster rate. Changing dressings less frequently causes fewer traumas to the wound area due to adhesives or other damage.

Traditional wound dressings have disadvantages not conducive to wound healing. Some absorbent dressings do not provide for a moist environment. Some absorbent and topical wound dressings cannot manage wounds with high amounts of exudate. Some dressings provide only a weak barrier for bacterial control. Certain topical compositions can dehydrate if they are not covered, making them unsuitable for dry wounds. Many dressings need frequent replacement or application. Some wound dressings are not suitable for chronic wounds.

Some adherent wound dressings have an added disadvantage of disturbing the wound healing process and the integrity of the surrounding skin upon removal. Adherent dressings form a tight bond with the skin over time. Removal of such dressings can also be painful. This is a particular disadvantage for adherent wound dressings that require frequent changing. A balance must be achieved-a wound dressing must be sufficiently adherent to be securely maintained on a patient's body, but not so adherent that removal can disturb the healing process or damage the surrounding skin.

Ideally, a wound dressing should promote healing, provide protection for the wound, be impermeable to bacteria, allow for gaseous exchange, contain non-toxic and non-allergenic materials, reduce trauma on wound dressing changes, possess improved durability (lifetime) of the wound dressing to limit the number of wound dressing changes needed, conform to the wound, possess improved fluid uptake, retention and removal properties, and be capable of maintaining a high humidity (moistness) at the wound site. A wound dressing should also limit or avoid systemic absorption of therapeutic or other agents present in the dressing.

Significant research into wound dressings is ongoing as new information surfaces regarding useful vehicles, materials, and/or therapeutic agents that promote more efficient healing. In one aspect, efforts to improve wound dressings to reduce wound trauma upon dressing changes, improve durability and lifetime of the dressing, conform a dressing to a wound, improve fluid uptake, retention and removal properties, maintain a moist environment, and allow for a secure attachment to a patient's body have focused on research with regard to multilayered wound dressings. As a result of research, a number of layered wound dressings are known and described in the art, and the focus has been on the use of multiple layers that address disadvantages inherently associated with traditional wound dressings. A variety of layered wound dressings are disclosed and include, by way of example, U.S. Pat. No. 6,043,408 directed to a wound dressing having a cover which is reversibly movable to an open position exposing the wound from a closed position covering the wound, thereby enabling selective viewing of the wound without removing the wound dressing.

U.S. Pat. No. 6,238,691 is directed to a hydrogel wound dressing comprising a polyurethane hydrogel composition capable of absorbing moisture from a wound site, which provides for moist wound healing, absorption of exudate, fewer dressing changes, and easy removal.

U.S. Pat. No. 6,346,653 is directed to a multilayer thin film, polyurethane dressing with an absorbent pad, having two grip tabs, one to facilitate removal of the dressing from a carrier layer and the other to facilitate removal of a cover sheet leaving the thin film dressing intact on the skin.

U.S. Pat. No. 6,903,243 discloses a multilayer wound dressing comprising at least two absorbent layers having different absorbencies, with the layer closest to the wound having a lower absorbency than the layer furthest from the wound. The dressing also contains additional non-absorbing layers, such as a backing film and a wound facing film. The dressing provides absorbency of wound exudates, while preventing absorbent material from entering the wound.

U.S. Pat. Nos. 7,223,899 and 7,154,017 are directed to a wound dressing and method for making a wound dressing, the wound dressing including an absorbent core defining opposed proximal and distal surfaces with at least one receptacle opening at the distal surface and extending into the thickness of the absorbent core, the at least one receptacle containing a discrete portion of absorbent material.

U.S. Pat. No. 7,214,847 and U.S. Pub. No. 2004/0049145 are directed to a multilayer conductive wound dressing, with the conductive material being silver.

U.S. Pat. No. 6,087,549 also discloses a multilayer laminate wound dressing comprising a plurality of layers of silver or silver-coated fibers alternating with layers of non-conductive fibers.

U.S. Pat. No. 8,021,347 is directed to a thin film wound dressing that includes a substantially transparent dressing having a pressure sensitive adhesive for applying the dressing over a wound to define a reservoir in which negative pressure may be maintained. A vacuum port is centrally located on the dressing layer adapted to provide fluid communication between a vacuum source and the reservoir. Vacuum drainage tubes or drains are also disclosed in U.S. Publication No. 2011/0301556.

U.S. Pat. No. 8,093,445 is directed to a wound dressing having multiple layers comprising a vapor permeable backing layer, a skin-adherent layer formed from a silicone gel elastomer, and absorbent core, and a covering layer.

Multilayer wound dressings impregnated with therapeutic (active) agents are known. Generally, in addition to multiple layers that include an adhesive layer, an absorbent layer, a film, and/or a cover or backing, a layer comprising therapeutic (active) agents may be provided. Various therapeutic agents provided in multilayered dressings for local effects include antibacterials, antifungals, antivirals, anti-infectives, antiseptics, antibiotics, wound healing promoting agents, anti-inflammatory agents, steroids, local analgesic or anesthetic agents, humectants, emollients, skin protection agents, and the like. By way of example, dressings having silver salts and certain antibiotics are known. U.S. Pat. Nos. 8,283,513 and 8,293,964 are directed to a flexible multilayer wound dressing having antibacterial and antifungal properties comprising a layer of silver containing fabric. U.S. Pat. No. 5,419,913 describes a laminate structure wound dressing having a hydrogel adhesive, wherein medicaments such as cetyl pyridium chloride and benzyl ammonium bactericides may be incorporated or bonded to the hydrogel polymer along the non-adhesive portion of the dressing to provide a slow-release mechanism for the therapeutic agent. As another example, a multilayered controlled release topical patch containing tetracycline for a wound dressing has been reported by G. Kiaee et al., J. of Silico & In vitro Pharmacology, Vol. 2, No 1:9, 2016, pp. 1-7 (also available at http://pharmacology.imedpub.com).

Wound dressings may also take the form of topical compositions containing therapeutic agents, which are applied directly to the skin. These topical dressings may or may not be covered with a material such as a fabric, film, foam, or bandage. By way of example, certain topical vehicles are known for providing an occlusive moist dressing and maintaining the therapeutic agent at the dermal level to promote local healing. These vehicles are used as carrier systems to aid in the delivery of an active therapeutic agent across the stratum corneum and into deeper regions of the skin, namely the epidermis and dermis, while minimizing or avoiding absorption into the systemic circulation.

For all wound dressings that comprise therapeutic agents, it is desirable to improve the contact of the therapeutic agent with the wound to take advantage of local effects while significantly reducing or eliminating the systemic absorption of the agent. The therapeutic agent desirably should be provided in a sustained release form or introduced through a device, vehicle, mechanism or material that slows release, so as to provide a continual release at the wound site over time, avoid or reduce systemic absorption, and limit the number of required wound dressing changes.

### Cannabinoids

Recently, important wound healing characteristics have been observed for cannabidiol (CBD). CBD is a non-psychoactive cannabinoid isolated from the Cannabis sativa plant, which has attracted great attention due to several beneficial effects in the literature. CBD has been investigated for the treatment of several neuropsychiatric disorders and diseases. It is known that CBD can help regulate the endocannabinoid system and reduce the release of excitatory neurotransmitters that can result in a retrograde inhibitor signal that lessens chronic pain responses. Research suggests that CBD can also reduce anxiety, facilitate restful sleep, inhibit inflammatory and neuropathic pain, and lower the craving for opiates.

Based on its ability to modulate the skin inflammatory response, CBD has been investigated in the treatment of several dermatological conditions, including psoriasis, atopic dermatitis, and acne. As one example, U.S. Patent Publication No. 2020/0000765 discloses the use of cannabinoid compounds, or other agents that work on the endocannabinoid system, in combination with polyprepolymers and stem cells for the treatment of skin disorders.

CBD has also been investigated for antimicrobial properties. CBD has been found to act synergistically with bacitracin against Staphylococcus aureus and other Gram-positive bacteria, opening a novel approach to treat cutaneous infections. U.S. Patent Publication No. 2023/0082130 A1 discloses use of cannabinoid compounds to treat or prevent growth of Candida albicans. U.S. Pat. No. 11,207,291 discloses use of cannabinoids as antimicrobial agents that are applied topically or given orally, alone or in combination with other antimicrobials, anti-inflammatory agents, or probiotics.

It has been shown that CBD significantly facilitates the wound healing process by controlling inflammatory infiltration, promoting collagen deposition and formation of granulation tissue and facilitating the formation of blood vessels. These local effects are highly desirable for wound healing. It has also been suggested that cannabinoids have positive effects on bone formation and bone healing. See e.g., Z. Zheng et al., Biomaterials Advances, Vol. 134, March 2022; S. Biasi et al., Cannabis-Based Medicines as Novel Approach to Treating Wound-Related Pain, Pain Management, November 2020; B. Raphael-Mizrahi, et al., The Cannabinoids Effect on Bone Formation and Bone Healing, Curr. Osteoporos Rep., October 2020.

Incorporation of CBD into topical wound dressings is known. By way of example, U.S. Publication No. 2020/0376156 discloses methods of treating epidermal wounds via topical application of various cannabinoids in combination with antibiotics and other wound care products in a composition applied directly to the skin or present on a bandage or other material. U.S. Publication No. 2020/0352849 discloses augmented topical systems comprising CBD incorporated into topical skin care formulations, wound care dressings, biomaterial skin substitutes or skin adhesives, in combination with other agents, such as creams, ointments, emollients, sprays, tinctures, gels, anti-inflammatory products, antibacterials, antifungals, antivirals, skin rejuvenation products, analgesics, antiitch products, moisturizers, sunscreens, sunburn relief products, wound dressing products, antidandruff products, hair growth promoters, scar treatments and wound care agents.

Incorporation of CBD in transdermal delivery systems to deliver CBD systemically is also known. U.S. Pat. No. 11,285,117 discloses a transdermal delivery device and method for delivery of CBD systemically through a patch having a reservoir designed to improve the bioavailability of the CBD to a human subject. Similarly, U.S. Pat. No. 6,113,940 discloses a transdermal structure to deliver cannabis chemicals to the bloodstream. U.S. Publication No. 2019/0110981 discloses buccal patches and dermal patches designed to improve delivery of cannabinoids to the systemic circulation. U.S. Publication No. 2017/0071870 also discloses a transdermal delivery device for the delivery of CBD and related moieties to the bloodstream. All of these transdermal delivery systems described above are designed to deliver CBD through the skin to avoid the gastric system and the liver first-pass effect and reduce toxic side effects while increasing bioavailability of the compounds.

Preferably, wound dressings comprising therapeutic agents, whether in a multilayered structure or a topical composition applied directly to the skin, should provide an effective and sufficient amount of the agent to allow for uniform delivery over time to the wound site without the need for frequent dressing changes. With conventional dressings, once the therapeutic agent is reduced or depleted, the dressing requires changing even though it may be intact, and healing may be ongoing. There is an unmet need, therefore, for wound dressings that allow for replenishment, renewal, or replacement of a therapeutic agent without the need to remove the wound dressing frequently. There is also a need for wound dressings that deliver and retain the therapeutic agent locally at the wound site, while limiting or avoiding systemic absorption.

Novel wound dressings, comprising CBD as a therapeutic agent, have been developed that provide all of the desirable attributes of a wound dressing and allow for renewing or replenishing CBD without frequent removal of the dressing. The inventive wound dressings may also comprise other therapeutic agents in combination with CBD. In one aspect, the inventive dressing comprises a multilayer dressing having an adhesive portion for fixing the dressing to the skin and at least two other portions, one of which comprises silver or other traditional wound dressing compounds and another comprising the therapeutic agent CBD alone or in combination with other therapeutic agents. The layers (sections or portions) of the dressing are positioned adjacent to each other in the dressing, or one layer may surround the other. The layers are not positioned over top of each other, but rather form discrete sections or portions of the dressing in substantially the same plane.

The inventive dressings are also provided with at least one vent (port) positioned on a side of the dressing away from the wound side and extending directly into the therapeutic portion creating an opening whereby therapeutic agent(s) may be added or reintroduced, replenished or renewed in the dressing. Cartridges, vials, ampoules or other similar devices, comprising additional therapeutic agents and configured to connect to the vent, are also provided as a part of a kit with the multilayered dressing.

The inventive multilayer dressings may also include a polymeric film or other material facing the wound and providing a support structure for the wound compound (silver) and CBD portions and/or a top cover or backing material positioned over the layers of the dressing and farthest away from the wound site. The inventive multilayer dressing may also include other layers (portions) that include absorbent materials, other therapeutic agents, and other wound care compositions.

In another aspect of the invention, topical wound dressing compositions comprising CBD and, optionally, other therapeutic agents for direct application to the wound have been developed that provide a uniform release of therapeutic agents to the wound while reducing or eliminating systemic absorption. The inventive topical wound dressings provide for improved local effects to and through the skin and, hence, improved healing. These topical wound dressings may be formulated in a vehicle that absorbs exudate, maintains a moist wound environment, and keeps or retains the therapeutic agent locally at the wound site. They may be covered with a sheet or film to prevent drying out.

The devices, compositions and methods of the invention provide CBD directly to a wound, with minimal or no systemic absorption over time and fewer dressing changes than currently required using traditional wound dressings. The CBD may also be combined with other therapeutic agents including without limitation herbal or natural products, such as aloe, turmeric, and honey; local anesthetics; analgesics, antibiotics, anti-infectives, antiseptics, disinfectants, bactericides, anti-inflammatory agents, antifungals, antivirals, steroids, silver compounds and other wound healing promoting agents, antioxidants, humectants, emulsifiers, penetration enhancers, and other skin enhancing products. Other therapeutic agents will be known to one skilled in the art.

It is an object of the invention to provide novel wound dressings to facilitate wound healing comprising at least one therapeutic agent that is CBD.

It is another object of the invention to provide novel wound dressings to facilitate wound healing, which are devised or formulated to deliver and retain therapeutic agents locally at the wound site to avoid systemic absorption.

It is a further object of the invention to provide wound dressings that require less frequent changes as compared to traditional wound dressings.

Another object of the invention is to provide a multilayer wound dressing configured with at least one vent or port, through which therapeutic agents may be added or renewed, replaced or replenished to reduce or avoid the need to change the dressing.

Yet another object of the invention is to provide a topical wound dressing composition for direct application to the skin, comprising cannabidiol (CBD) alone or in combination with other therapeutic agents, incorporated into a vehicle that provides slow release of the therapeutic agent(s), maintains a moist environment, and delivers and maintains the therapeutic agent at the wound site while reducing or eliminating systemic absorption.

Still another object of the invention is to provide an absorbent multilayered wound dressing, comprising CBD alone or in combination with other therapeutic agents, whereby the CBD and other therapeutic agents may be added or replaced, replenished, or renewed without disturbing the wound dressing.

Other objects of the invention will be known to one skilled in the art based on the disclosure herein.

### SUMMARY OF THE INVENTION

The invention is directed to a multilayer wound dressing having improved wound healing properties, comprising an adhesive layer, a layer of silver or other traditional wound dressing compounds, and a therapeutic layer comprising cannabidiol (CBD) alone or in combination with other therapeutic agents. The layers are not stacked atop each other, but rather are adjacent to each other or configured around each other in substantially the same plane. The inventive wound dressing includes at least one vent (or port) positioned on an outer side of the dressing away from the wound and extending into and in direct communication with the therapeutic layer, to provide an opening through which cannabidiol or other therapeutic agents may be added or replaced, replenished or renewed without having to remove the dressing. The inventive wound dressings may also include a film or other material on the wound side (“wound facing”) as a support structure for the layers and to provide a fluid permeable barrier between the wound site and the layers. The inventive wound dressings may also include a top, outer cover, or backing material covering the layers of the wound dressing and positioned the farthest away from the wound.

The layers of the inventive multilayer wound dressings are not limited in number or in position vis a vis each other, except that the adhesive layer is positioned around the outer edge of the dressing. Additional layers, such as an absorbent material layer, may also be included and are within the scope of the invention. Alternatively, the backing material or wound facing material may comprise or incorporate absorbent materials.

The invention is also directed to a topical wound dressing compositions for direct application to the skin, comprising CBD alone or with other therapeutic agents in a vehicle that provides a wound barrier, maintains a moist environment, and limits or prevents systemic absorption.

In one embodiment, the invention is a wound dressing kit, comprising:
a. a multilayer wound dressing comprising:
   i. an adhesive layer for fixing the dressing to the skin;
   ii. at least one inner layer containing a wound care composition;
   iii. another inner layer comprising at least one therapeutic agent that is CBD; and
   iv. at least one port positioned on an outer side of the dressing away from the wound and extending into and in direct communication with the therapeutic layer; and
b. a cartridge containing therapeutic agents configured to connect to the port for addition or replenishment of the therapeutic agents.

In another embodiment, the invention is a multilayer wound dressing comprising:
a. an adhesive layer;
b. at least one layer of absorbent material;
c. a therapeutic layer comprising at least one therapeutic agent that is cannabidiol;
d. a layer comprising silver or other wound care compound;
e. optionally, a film or other material facing the wound and serving as a support structure for the adhesive, absorbent and therapeutic layers;
f. optionally, a top or backing material positioned farthest away from the wound and covering the layers of the multilayer wound dressing;
g. at least one port positioned on an outer side of the dressing away from the wound and extending into the dressing to allow for addition or replacement of cannabidiol or other therapeutic agents.

In yet another embodiment, the invention is a wound dressing comprising:
a. a multilayer wound dressing comprising:
   i. an adhesive layer,
   ii. at least one inner layer comprising silver or other wound care compositions;
   iii. another inner therapeutic layer comprising cannabidiol and at least one therapeutic agent selected from the group consisting of antibiotics, anti-infectives, antiseptics, antifungals, antivirals, anti-inflammatory agents, analgesics, anesthetics, skin care agents, and absorption enhancers; and
   iv. at least one port positioned on an outer side of the dressing away from the wound and extending into the wound dressing to allow for replacement or addition of therapeutic agents.

Still another embodiment, the invention is a topical composition for direct application of the skin comprising CBD alone or in combination with other therapeutic agents, incorporated into a vehicle that delivers and maintains the CBD or other therapeutic agents at the wound side and prevents systemic absorption.

Still other embodiments will be evident to one skilled in the art based on the disclosure herein.

### BRIEF DESCRIPTION OF THE DRAWINGS

FIG. 1 shows an example of an embodiment of the inventive multilayer wound dressing viewed from the top. Element 1 of FIG. 1 is an innermost layer comprising silver or other traditional wound dressing compound. Element 2 of FIG. 1 is a therapeutic layer incorporating CBD with or without other therapeutic agents surrounding element 1 in the same plane. Element 3 of FIG. 1 is an adhesive layer positioned on the outer edges of the multilayer wound dressing and surrounding elements 1 and 2. Element 4 of FIG. 1 is an outside opening of vent(s) positioned on the outer side of the dressing away from the wound for adding or replenishing the therapeutic layer of the dressing with additional CBD or other therapeutic agents. Element 5 of FIG. 1 is a portion of the vent extending into and in direct communication with the therapeutic layer (element 3) through which therapeutic agents may be added or replenished.

FIG. 2 (6) is a CBD-containing vial, cartridge or ampoule configured to connect with element 4.

FIG. 3 is a side view of the inventive multilayer dressing. Element 7 is the adhesive layer (element 3) viewed from the side. Element 8 is the therapeutic layer (element 2) from the side. Element 9 is a cross section of element 4 (the outside opening of the vent). Element 10 is the cross section of the vent (element 5) (side view) extending an opening into the therapeutic layer (element 8).

### DETAILED DESCRIPTION OF THE INVENTION

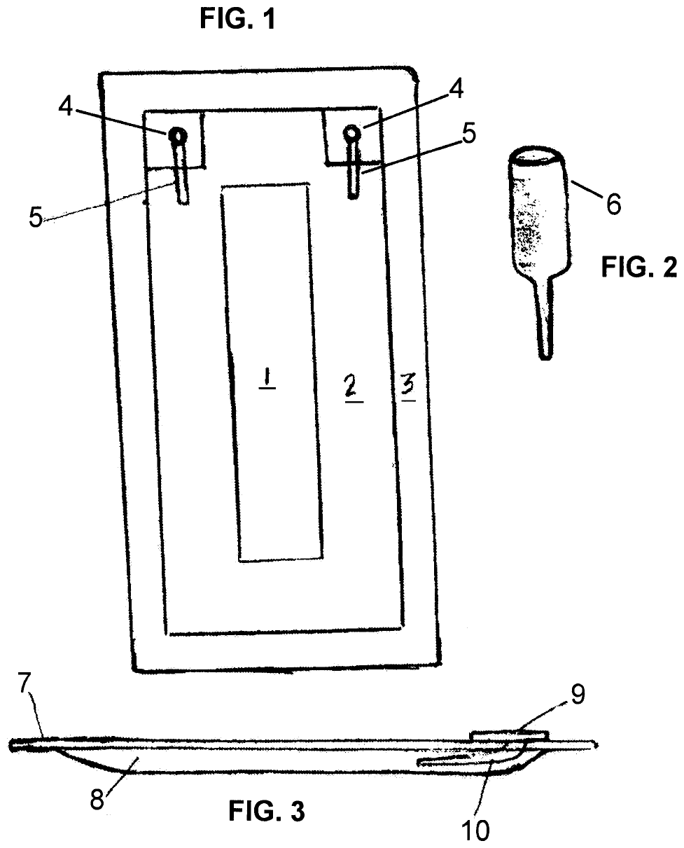

The invention is directed to a multilayer wound dressing having improved wound healing properties, comprising an adhesive layer (3) (7), a layer of silver or other traditional wound dressing compounds (1), and a therapeutic layer (2) (8) comprising cannabidiol (CBD) alone or in combination with other therapeutic agents. The layers are not stacked atop each other, but rather are adjacent to each other or configured around each other in substantially the same plane as shown in FIG. 1. The inventive wound dressing includes at least one vent (or port) (4) (9) positioned on an outer side of the dressing away from the wound and extending into and in direct communication with the therapeutic layer, to provide an opening (5) (10) through which cannabidiol or other therapeutic agents may be added or replaced, replenished or renewed without having to remove the dressing. The inventive wound dressings may also include a film or other material on the wound side (“wound-facing”) as a support structure for the layers and provides a fluid permeable barrier between the wound site and the layers of the multilayer dressing. The inventive wound dressings may also include a top, outer cover, or backing material covering the layers of the wound dressing and positioned the farthest away from the wound.

The layers of inventive wound dressings disclosed herein are not limited in number or in position vis a vis each other, except that the adhesive layer is positioned around the outer edge of the dressing. It will be appreciated that additional layers, such as an absorbent material layer, are within the scope of the invention. Alternatively, the backing material or wound facing material may comprise or incorporate absorbent materials.

The invention is also directed to a topical wound dressing compositions for direct application to the skin, comprising CBD alone or with other therapeutic agents in a vehicle that provides a wound barrier, maintains a moist environment, facilitates healing, and limits or prevents systemic absorption of the therapeutic agent.

A kit comprising the inventive multilayer wound dressing and cartridges (vials or ampoules) (FIG. 2 (6)) for replacement of therapeutic agents is also contemplated by the invention.

As used herein, “layer” means a functional section, part, or portion of the multilayer dressing positioned adjacent to other sections, parts, or portions of the dressing in the same plane and not atop each other. “Layer”, “section” and “portion” are used interchangeably herein.

“Vent” means a port, valve, or other structure, which provides an opening through which therapeutic agents may be added and/or replenished in the therapeutic layer. “Vent” and “port” are used interchangeably herein.

“Backing”, “top”, or “outer cover” are used interchangeably to describe a material positioned over the layers of the multilayer dressing and positioned the farthest away from the wound or wound side.

“Cartridge”, “ampoule”, and “vial” are used interchangeably herein and describe any container comprising CBD or other therapeutic agent, which is adapted or configured to connect to the vent.

Delivering drug molecules to and through the skin involves a complex interplay between the active drug, the type of delivery system or vehicle, the choice of excipients, skin type and location, and skin condition. The delivery system may be a device or a vehicle compound that incorporates and transports an active drug or therapeutic agent into contact with and through the skin at an appropriate level to provide a therapeutic effect. The challenge to topical drug delivery is the transport across the stratum corneum (SC). To overcome this barrier, a device or vehicle compound must maintain the solubility and stability of the active drug; release the active drug to the skin with even distribution; enable penetration into and permeation through the SC skin barrier; facilitate partitioning from the SC into and diffusion through the viable epidermis; sustain the active drug at the target site for a sufficient duration to provide a therapeutic effect; and limit or prevent systemic absorption. Furthermore, a device designed for delivery of a therapeutic agent should conform to the wound, be comfortable for the subject, and easy to apply and remove. A compound serving as a vehicle should be soothing and comfortable, spread easily and be aesthetically pleasant, which aids in patient compliance. These important attributes of a topical vehicle compound are dependent on the selection of the right excipients. See e.g., Barnes, T. M. et al., Vehicles for Drug Delivery and Cosmetic Moisturizers: Review and Comparison, Pharmaceutics 2021, 13, 2012. https://doi.org/10.3390/pharmaceutics13122012. (“Barnes”). Many excipients have multiple properties and effects on the skin, which may also be leveraged to promote wound healing.

Intact SC is a major barrier for drug penetration as it is impermeable to most compounds and molecules with a molecular weight greater than 600 Daltons. In many wounds, the SC is not intact, thus allowing for more efficient permeation of the drug across the dermal layers. FDA-approved molecules for transcutaneous drug delivery are those with lower molecular weight (<500 Daltons) with balanced lipophilicity and hydrophilicity. These lower molecular weight compounds provide local pharmacological effects and systemic effects. CBD has a molecular weight of 314.5 g/mol (Daltons). This low molecular weight and highly lipophilic nature of CBD make it an ideal molecule for administration via the transdermal route, thus avoiding the low bioavailability associated with the oral route. A wide variety of currently available dosage forms exist for CBD, the vast majority of which facilitate systemic effects.

The challenge for use of CBD in wound dressings is to provide for local, not systemic, effects. CBD must be provided in a wound dressing where the therapeutic agent will be well absorbed into the skin but have limited or no systemic effects. In this regard, CBD and other therapeutic agents are provided to the inventive multilayered wound dressing in a carrier or slow-release form or incorporated into a topical wound dressing formulated with a vehicle that delivers and maintains the CBD and other therapeutic agents locally at the wound site.

A preferred embodiment of the invention is a multilayer wound dressing comprising an adhesive layer (3) adjacent to and surrounding a therapeutic layer containing CBD (2), which in turn surrounds an innermost layer containing a silver-based wound care compound (1). All of the layers are configured spatially on substantially the same plane of the dressing.

It will be appreciated that the invention may comprise other layers including a layer of absorbent materials and that the layers may be positioned differently within the adhesive layer depending on the nature of the wound. The inventive dressing may also include a wound-facing layer that serves as a support structure for the other layers and provides a fluid permeable barrier between the wound site and the layers of the multilayered dressings. The inventive dressing may also include a water resistant or water impermeable backing or outer cover positioned farthest from the wound site. The invention may also comprise additional therapeutic agents in combination with CBD. Therapeutic agents are provided in slow-release form or incorporated in a carrier that facilitates slow release, so as to limit or avoid systemic absorption of the therapeutic agent.

### Materials for Multilayered Wound Dressings

Various materials are suitable for forming the multilayered wound dressing of the invention. Absorbent materials include without limitation cotton, gauze, woven and non-woven materials, cloth, fabrics, polymeric foams, sponges, polymeric films, polymeric particulates, alginates and other polysaccharide materials, carboxymethylcellulose (CMC), silicones, hydrophilic polymers, monomeric materials, hydrogels, hydrocolloids, and the like. A plethora of absorbent materials are available for use in wound care dressings that claim to have the ability to manage exudate while facilitating healing. Many materials are suitable not only as absorbent material, but also as wound-facing sheets or films and backing materials. Some absorbent materials may also have antimicrobial properties.

Suitable polymers for use in the absorbent layer, a wound-facing sheet or film, or backing material include without limitation polyurethanes, polyamides, polyesters, polyethylenes, and polypropylenes. Other suitable materials will be known to one skilled in the art.

The backing material, when used, should be conformable to the anatomical surface on which the dressing is to be used and highly resistant or impermeable to liquids. The backing layer can be transparent and elastomeric. Preferably, the backing material will allow some moisture to evaporate from the dressing while still maintaining a moist environment. The backing material may comprise or incorporate absorbent materials. Suitable materials for the backing material include without limitation papers, non-woven fabrics, natural fibers and fabrics, cotton, synthetic fabrics, synthetic films and foams, polymeric materials, including the polymers described above, mesh, woven fabrics, and knit fabrics. A wide variety of backing materials are known to one skilled in the art.

Adhesive materials for use in the adhesive layer of the multilayer wound dressing are known in the art. Adhesive materials should stick well to human skin and be capable of being removed from that same skin. Four factors need to be considered in selecting the adhesive: the application, the skin type, the location, and length of wear. Preferably, adhesives are non-toxic and non-allergenic. Adhesives may be formulated as pressure sensitive adhesives to facilitate easy application. Suitable adhesives may be silicone-based. Silcones are soft and work well for sensitive skin applications because they stick well and are easy to remove. Acrylic adhesives provide more adhesion than silicones and are commonly used in pressure sensitive formulations. Rubber adhesives are the strongest and stay on the longest. Hydrocolloids are gel-like adhesives that provide non-breathable adhesion to the skin and trap moisture under the skin, thus promoting healing in wound care products. In some instances, depending on the material used, the wound-facing film or sheet may also double as an adhesive for the multilayer dressing.

Wound care compounds include silver compounds, such as silver chloride, silver fibers, silver particles, and nanoparticle silver. Silver compounds provide antibacterial, anti-inflammatory, and antioxidative properties that facilitate healing and have long been known to be useful compounds for wound dressings. Natural compounds are also suitable for use in wound care and include without limitation plant and herbal compounds, such as aloe vera, Calendula officinalis, Hippophae rhamnoides (sea buckthorn), Angelica sinensis, Catharanthus roseus, Sesamum indicum and Morinda citrifolia; polysaccharides; glucomannan; lectins; and animal-derived products such as honey and propolis.

Antiseptics are also useful wound care compounds, and many provide a broad-spectrum antimicrobial effect. Useful antiseptics may be included in the wound care compound layer, alone or in combination with silver compounds, or in the therapeutic layer in combination with CBD. Suitable antiseptics include povidone-iodine, chlorhexidine, certain peroxides, boric acid, chlorides, ammonium salts, triclosan, hexachlorophene, and quaternary ammonium compounds.

The inventive wound dressings include a therapeutic agent, CBD, alone or in combination with other therapeutic agents. Other therapeutic agents include antibiotics, such as amikacin, gentamicin, clindamycin, bacitracin, neomycin, polymyxin, and metronidazole; local anesthetics, such as benzocaine and lidocaine; anti-inflammatory agents, such as diclofenac, pain relief agents, such as capsaicin; antiseptics, such as povidone-iodine, chlorhexidine, triclosan, benzalkonium chloride, and quaternary ammonium compounds; antifungals, such as nystatin, ketoconazole, terbinafine, miconazole, tolnaftate, clotrimazole, and the like; and antivirals, such as acyclovir; steroids, such as hydrocortisone, triamcinolone, mometasone, clobetasol, fluocinonide, desonide, and the like. Other therapeutic agents will be known to one skilled in the art. Therapeutic agents of the inventions preferably are provided in slow-release form or are incorporated in a carrier or vehicle that facilitates slow release, so as to limit or avoid systemic absorption.

### Vehicles for Topical Wound Compositions

Various topical vehicles may be used as carrier systems to add in the delivery of active drug across the SC and into deeper regions of the skin, while minimizing or avoiding systemic absorption. Topical vehicles are selected based on the nature (wet/dry, mucous/non-mucous, health/diseased) and size of the skin area to be treated. See Barnes, T. M., supra. The most common topical vehicles include semi-solid ointments, creams, gels, salves, and pastes and liquid lotions, solutions, foams and sprays.

Ointments provide a barrier to air and external contaminants, are resistant to water and other liquids, and improve hydration, transepidermal water loss, and skin softness and elasticity, while limiting systemic effects significantly. Polysaccharide cryogels containing B-cyclodextrin and other polysaccharide molecules are known to provide slow release of CBD to skin malignancies. Composite materials, emulsions stabilized with chitosan/collagen peptides nanoparticles, microemulgels liposomes, micelles, oils, and polymeric nanoparticles, among others, have also been studied as potential cutaneous delivery of cannabinoids, including CBD. Other suitable vehicles include without limitation liquid paraffin, virgin olive oil, propylene glycol, purified water, PEG 400, white petrolatum, cetostearyl alcohol, cetomacrogol 1000, hydroxyethyl cellulose, Eudragit® RL, glycerin, ceramides, fatty alcohols, squalene, lactic acid, glycolic acid, polyhydroxy acids, such as gluconolactone and lactobionic acid, tributyl citrate, liposomes, tyrosine derived nanospheres, beeswax, cocoa butter, coconut oil, or safflower, pumpkin seed or grapeseed oils.

### Dosage/Concentrations

CBD dosages vary. For the purposes of the multi-layered wound dressings, effective amounts of CBD are present in the therapeutic layer in amounts ranging from about 10 mg. to about 100 mg. Since the invention contemplates adding or replenishing CBD in the therapeutic layer, dosages may be adjusted over time or for effect. Preferably, the CBD is provided in the therapeutic layer in a slow-release form or incorporated in a slow-release carrier system.

Suitable concentrations of CBD in a topical skin formulation range from about 1% w/w to about 20% w/w based upon the total weight of the topical formulation, with the amount depending on the vehicle selected.

Suitable concentrations for therapeutic agents, whether applied through a multilayered wound dressing or a topical formulation directly to the skin, are known. By way of example, the topical anesthetic lidocaine may be utilized in the inventive dressings in amounts ranging from about 0.5% to 5% based on the type and weight of the carrier. Anti-inflammatories, such as diclofenac, may be incorporated into wound dressings in amounts ranging from 1% to 3% based on the type and weight of the carrier. Antibiotics such as amikacin are available in gels or cream carriers in amounts of 5% based on the total weight of the composition. Clindamycin, another antibiotic, may be provided in amounts of 1%-2% concentration based on the total weight of the composition. Topical minocycline is available in a topical foam in a 1.5% or 4% concentration. Azelaic acid which has both anti-inflammatory and antimicrobial properties may be incorporated into a wound dressing in amounts ranging from 10%-20% based on the weight of the composition. Other dosages and concentrations of therapeutic agents applied through a topical device or composition are known in the art.

Optional excipients may be incorporated into the inventive wound dressings to soothe the skin, enhance skin protection, or promote absorption including without limitation, exfoliants, emulsifiers, humectants, moisturizers, Vitamin C, Vitamin E, skin protection agents, skin feel agents, collagen promoting agents, or mixtures thereof. Preservatives may also be incorporated depending on the sensitivity of the skin to be treated. Other excipients will be known to one skilled in the art.

The inventions are further disclosed in FIGS. 1-3 and in the non-limiting examples set forth herein.

### EXAMPLE 1

### Surgical Dressing Kit

A surgical dressing kit is prepared to include a multilayer wound dressing comprising: an adhesive layer for adhering the dressing to the skin, a wound dressing layer comprising one or more traditional wound care compounds, a therapeutic layer containing CBD, and a backing or outer cover. The wound dressing compound is a silver compound. The adhesive layer surrounds the therapeutic layer, which in turn surrounds an innermost layer comprising the traditional wound care composition. The layers of the multilayer wound dressing are positioned in substantially the same plane.

CBD is provided in a slow-release form such as an ointment, gel, polymer matrix or paste in amounts of at least 20 mg. The backing or outer cover is a water resistant or water impermeable material. The multilayer dressing is constructed with at least one port (vent) positioned on the backing or outer cover which extends into and is in communication with the CBD therapeutic layer. The port provides an opening for addition or replenishment of CBD without the need for frequent dressing changes.

The surgical dressing kit also includes a cartridge or ampoule containing CBD, which is adapted or configured to connect to the port and provides direct communication with the therapeutic layer so that CBD may be replenished in the therapeutic layer.

### EXAMPLE 2

### Wound Dressing Kit

A wound dressing kit is prepared the same as the surgical dressing kit of Example 1. The multilayer wound dressing is the same as the multilayer wound dressing of Example 1 except that additional therapeutic agents are included in the therapeutic layer comprising diclofenac alone or in combination with lidocaine. The diclofenac is provided as a 1% or 3% topical ointment, cream, gel, or paste. The lidocaine is provided as a 3%-5% gel, cream, ointment, or paste.

### EXAMPLE 3

### Absorbent Multilayer Dressing

An absorbent multilayer wound dressing is prepared the same as the multilayer wound dressing of Example 1, except that it includes an additional layer comprising an absorbent material.

### EXAMPLE 4

### Absorbent Multilayer Dressing

An absorbent multilayer wound dressing is prepared the same as the multilayer wound dressing of Example 2, except that it includes an additional layer comprising an absorbent material.

### EXAMPLE 5

### Multilayer Wound Dressings

Multilayer wound dressings are prepared the same as the multilayer wound dressings of Examples 1, 2, 3 or 4, except that they include a wound facing film or sheet as a support structure for the layers of the multilayer wound dressings.

### EXAMPLE 5

### Multilayer Wound Dressings

Multilayer wound dressings are prepared the same as the multilayer wound dressings of Examples 1, 2, 3, 4 or 5, except that they include additional wound care compounds in combination with the silver compound, comprising antiseptics.

### EXAMPLE 6

### Topical Wound Dressing

A topical wound dressing is provided comprising a wound care composition, CBD, and one or more of diclofenac and lidocaine. The CBD is provided in amounts of 1%-20% based on the total weight of the topical formulation, diclofenac is provided in amounts ranging from 1%-3% based on the total weight of the topical formulation, and lidocaine is provided in amounts ranging from 3%-5% based on the total weight of the topical formulation. The vehicle for the topical wound care dressing is a paste comprising an ointment with insoluble solids as is known in the art.

### EXAMPLE 7

### Topical Wound Dressing

A topical wound dressing is provided as in Example 6, except the vehicle for the topical dressing is a polymeric matrix comprising natural or synthetic gums.

### EXAMPLE 8

### Topical Wound Dressing

Multiple topical wound dressings are provided as in Example 6, wherein the following vehicles are used: natural or synthetic polymeric nanoparticles, liposomes, tyrosine derived nanospheres, beeswax, cocoa butter, coconut oil, petrolatum, or olive, safflower, pumpkin seed or grapeseed oils.

In accordance with the patent statues, the best mode and preferred embodiment have been set forth. The scope of the invention is not limited thereto, but rather by the scope of the attached claims.

We claim:

1. A surgical wound dressing kit, comprising:
a. a multilayer wound dressing comprising:
i. an adhesive layer on the outer edge of the multilayer wound dressing for fixing the multilayer wound dressing to the skin;
ii. a therapeutic layer containing therapeutic agents comprising at least cannabidiol;
iii. a wound care composition layer comprising a silver compound;
iv. a backing or outer cover, and
v. at least one port positioned on the outer cover extending into and in direct communication with the therapeutic layer to provide an opening through which cannabidiol or other therapeutic agents, may be added or replenished; and
b. a cartridge containing additional cannabidiol or other therapeutic agents, configured to connect to the port to provide additional cannabidiol or other therapeutic agents,
wherein the layers of the multilayer wound dressing are positioned adjacent to or surrounding each other spatially in substantially the same plane on the dressing.

2. The surgical wound dressing kit according to claim 1, wherein the multilayer wound dressing further comprises an absorbent material.

3. The surgical wound dressing kit according to claim 1, wherein the therapeutic layer comprises additional therapeutic agents in combination with cannabidiol.

4. The surgical wound dressing kit according to claim 3, wherein the additional therapeutic agents comprise a local anesthetic, an analgesic, an anti-inflammatory, or an antibiotic, or mixtures thereof.

5. The surgical wound dressing kit according to claim 4, wherein the local anesthetic is lidocaine and wherein the anti-inflammatory is diclofenac.

6. The surgical wound dressing kit according to claim 3, wherein the therapeutic agents comprise cannabidiol, lidocaine and diclofenac.

7. A topical wound dressing comprising:
a. a vehicle for carrying therapeutic agents for application to the skin, wherein the vehicle maintains therapeutic agents at the wound site and reduces or eliminates systemic absorption of the therapeutic agents;
b. a therapeutic agent that is cannabidiol (CBD);
c. optionally, additional therapeutic agents comprising antibiotics, anti-inflammatories, antifungals, analgesics, or local anesthetics, or mixtures thereof;
d. optionally, wound healing promoting agents; and
e. optionally, excipients.

8. The topical wound dressing according to claim 7, wherein the vehicle is white petrolatum, propylene glycol, PEG 400, liquid paraffin, oil, or mixtures thereof.

9. The topical wound dressing according to claim 7, wherein the vehicle is selected from the group consisting of: virgin olive oil, cetostearyl alcohol, cetomacrogol 1000, hydroxyethyl cellulose, ceramides, fatty alcohols, squalene, lactic acid, glycolic acid, polyhydroxy acids, tributyl citrate, liposomes, tyrosine derived nanospheres, beeswax, cocoa butter, coconut oil, safflower oil, pumpkin seed oil or grapeseed oil.

10. The topical wound dressing according to claim 7, wherein the excipients comprise emulsifiers, humectants, antioxidants, or preservatives, or mixtures thereof.

11. The topical wound dressing according to claim 7, wherein cannabidiol (CBD) is present in the dressing at a concentration of about 1% w/w to about 20% w/w based on the total weight of the topical wound dressing.

12. The topical wound dressing according to claim 7, wherein the additional therapeutic agents comprise benzocaine, lidocaine, diclofenac, or mixtures thereof.

13. A multilayer wound dressing comprising:
a. an adhesive layer for fixing the multilayer wound dressing to the skin;
b. a therapeutic layer comprising at least one therapeutic agent that is cannabidiol;
c. a wound care composition layer comprising a silver compound;
d. a backing or outer cover positioned over the layers and farthest from the wound site, and
e. at least one port positioned on the outer cover extending into and in direct communication with the therapeutic layer to provide an opening through which therapeutic agents may be added or replenished,
wherein the layers of the multilayer wound dressing are positioned adjacent to or surrounding each other in substantially the same plane on the dressing.

14. The multilayer wound dressing of claim 13, further comprising an absorbent material.

15. The multilayer wound dressing of claim 13, further comprising a wound facing film positioned between the wound site and the layers.

16. The multilayer wound dressing of claim 13, wherein the therapeutic layer comprises therapeutic agents in addition to cannabidiol.

17. The multilayer wound dressing according to claim 16, wherein the additional therapeutic agents comprise lidocaine and diclofenac.

18. The multilayer wound dressing according to claim 13, wherein the silver compound comprises silver nanoparticles.

19. The multilayer wound dressing according to 17, wherein the silver compound comprises silver nanoparticles.
